(19) United States
(12) Patent Application Publication
(10) Pub. No.: US 2026/0232551 A1
(43) Pub. Date: Aug. 13, 2026

(54) COMPOSITIONS SUITABLE FOR USE ON SENSITIVE SKIN AND METHODS USING SAME

(71) Applicant: Kenvue Brands LLC, Summit, NJ (US)

(72) Inventors: Simarna Kaur, Neshanic Station, NJ (US); Neena Tierney, Summit, NJ (US)

(21) Appl. No.: 19/147,186

(22) PCT Filed: Jan. 10, 2024
(86) PCT No.: PCT/IB2024/050259

§ 371 Date: Jul. 10, 2025

Related U.S. Application Data
(60) Provisional application No. 63/479,392, filed on Jan. 11, 2023.

(51) Int. Cl. A61K 8/36; A61K 8/9789; A61P 17/04; A61P 17/06; A61Q 19/00

(52) U.S. Cl. CPC A61K 8/361; A61K 8/9789; A61P 17/04; A61P 17/06; A61Q 19/005

(57) ABSTRACT

Provided are compositions and methods of treating sensitive skin with compositions comprising compounds having retinol-like activity. Also provided are methods of treating acne, wrinkles and/or lightening skin in a subject having a reaction condition or a history of a reaction condition with compositions comprising compounds having retinol-like activity.

### FIELD

The present invention generally relates to compositions suitable for use on sensitive skin and particularly methods of using the compositions to treat the skin.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned ageing processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, despite the beneficial effects of retinoid treatment, its benefits are limited due to skin irritation of retinoids. These side effects can restrict the use of retinoids, and particularly so for individuals having sensitive skin.

To date, the search for alternative compounds to replace retinoids, and particularly for individuals having sensitive skin, has produced limited success in treating skin conditions associated with aging, such as skin atrophy, acne, photo-aging, and in reducing the appearance of wrinkles, fine lines, stretch marks, or cellulite.

Accordingly, there is a need for alternatives to traditional retinoids which are efficacious but gentle enough for use on individuals having sensitive skin.

### SUMMARY

Accordingly, one aspect of the invention pertains to a method of treating sensitive skin, the method comprising applying to the sensitive skin a compound of Formula I:

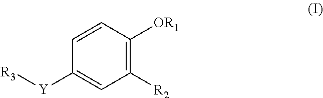

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and
   Y is —(CH₂—CH₂)—, —(CH═CH)—, or —(C═C)—;
or a cosmetically acceptable salt thereof.

In one or more embodiments, R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)—. In some embodiments, R₁ is selected from the group consisting of C₅-C₁₆ alkenyl; and R₂ is selected from the group consisting of hydrogen or —OC₁-C₃ alkyl. In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In some embodiments, the compound of Formula I is 3-(4-farnesyloxyphenyl)-propionic acid. In one or more embodiments, the applying step comprises applying to the sensitive skin the compound of Formula I, and the compound of Formula I is contained in a botanical extract. In some embodiments, said botanical extract is an extract of a plant of the genus Acronychia. In one or more embodiments, said botanical extract is an extract of Acronychia acidula. In some embodiments, said extract is a polar extract. In one or more embodiments, the extract is present in an amount of about 0.1 to about 3 wt. % by total weight of the composition. In some embodiments, the extract is present in an amount of about 0.4 to about 1.5 wt. % by total weight of the composition. In one or more embodiments, the sensitive skin comprises skin of a subject who has a reaction condition or a history of a reaction condition selected from the group consisting of erythema, edema, burning, pain, pruritis, stinging, tingling, itching, tightness feeling, dry feeling, scaling, tactile roughness, peeling skin, flaky patches and combinations thereof when exposed to a retinoid. In some embodiments, the sensitive skin comprises skin of a subject who has a condition or a history of a condition selected from the group consisting of atopic dermatitis, rosacea, seborrheic dermatitis, contact dermatitis, psoriasis, dry skin, flaky skin. In one or more embodiments, the method of treating skin is a method of treating acne, wrinkles, and/or lightening skin in a subject who has a condition or a history of a condition selected from the group consisting of atopic dermatitis, rosacea, seborrheic dermatitis, psoriasis, dry skin, flaky skin.

Another aspect of the invention pertains to a method of treating acne, wrinkles and/or lightening skin in a subject having a reaction condition or a history of a reaction condition selected from the group consisting of erythema, edema, burning, pain, pruritis, stinging, tingling, itching, tightness feeling, dry feeling, scaling, tactile roughness, peeling skin, flaky patches, and combinations thereof, the method comprising applying to the skin 3-(4-farnesyloxyphenyl)-propionic acid.

In some embodiments, the applying step comprises applying to the sensitive skin a composition comprising a botanical extract is an extract of a plant of the genus Acronychia. In one or more embodiments, said botanical extract is an extract of Acronychia acidula. In some embodiments, said extract is a polar extract. In one or more embodiments, the extract is present in an amount of about 0.1 to about 3 wt. % by total weight of the composition. In some embodiments, the extract is present in an amount of about 0.4 to about 1.5 wt. % by total weight of the composition.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a composition that is “essentially free” of an ingredient means the composition that has about 2% or less of that ingredient by weight based on the total weight of the composition. Preferably, a composition that is essentially free of an ingredient has about 1% or less, more preferably about 0.5% or less, more preferably about 0.1% or less, more preferably about 0.05 or less, more preferably about 0.01% or less by weight based on the total weight of composition of the ingredient. In certain more preferred embodiments, a composition that is essentially free of an ingredient is free of the ingredient, i.e. has none of that ingredient in the composition.

As used herein, “cosmetically/dermatologically acceptable” means that the ingredients which the term describes are suitable for use in contact with tissues (e.g., the skin or hair) without undue toxicity, incompatibility, instability, irritation, allergic response, and the like. As will be recognized by one of skill in the art, cosmetically/dermatologically acceptable salts are acidic/anionic or basic/cationic salts.

As used herein, the term “safe and effective amount” means an amount of the extract or of the composition sufficient to induce the desired effect, but low enough to avoid serious side effects. The safe and effective amount of the compound, extract, or composition will vary with e.g. the age, health and environmental exposure of the end user, the duration and nature of the treatment, the specific extract, ingredient, or composition employed, the particular pharmaceutically-acceptable carrier utilized, and like factors.

As used herein, the term “about” refers to within 5% weight, within 4% weight, within 3% weight, within 2.5% weight, within 2% weight, or within 1% weight of a disclosed value.

In general, IUPAC nomenclature rules are used herein and according to the following term definitions.

The term “substituted,” refers to a core molecule in which one or more hydrogen atoms have been replaced with that amount of substituents allowed by available valences. Substitution is not limited to the core molecule, but may also occur on a substituent radical, whereby the radical becomes a linking group.

The term “independently selected” refers to two or more substituents that may be selected from a substituent variable group, wherein the selected substituents may be the same or different.

The term “dependently selected” refers to one or more substituent variables that are specified in an indicated combination for substitution in a core molecule (e.g. variables that refer to groups of substituents appearing in a tabular list of compounds).

Acceptable salts from inorganic bases include, for example, sodium or potassium salts, and the like. Acceptable salts from organic bases include, for example, salts formed with primary, secondary, or tertiary amines, and the like.

One aspect of the invention pertains to methods of treating sensitive skin, the method comprising applying to the sensitive skin a compound of Formula I:

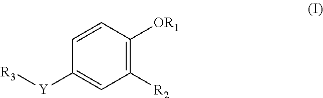

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and
   Y is —(CH₂—CH₂)—, —(CH═CH)—, or —(C═C)—;
or a cosmetically acceptable salt thereof.

### Compound of Formula I

In one or more embodiments, R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)— (or a cosmetically acceptable salt thereof). In some embodiments, R₁ is selected from the group consisting of C₅-C₁₆ alkenyl; and R₂ is selected from the group consisting of hydrogen or —OC₁-C₃ alkyl. In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In one or more embodiments, the compound of the above Formula I is 3-(4-farnesyloxyphenyl)-propionic acid and/or its ethyl ester. In preferred embodiments, the compound of Formula I is comprises 3-(4-farnesyloxyphenyl)-propionic acid. The 3-(4-farnesyloxyphenyl)-propionic acid and/or its ethyl ester can be synthesized using conventional organic synthesis processes.

The compound of Formula I may be present in an amount ranging from about 0.00001% to 10%, or about 0.0001 to about 10%, or about 0.001 to about 5%, or about 0.001% to about 1%, or about 0.01% to about 3%, about 0.01% to about 1%, about 0.01% to about 0.5%, or about 0.005% to about 1.5%, or about 0.005% to about 0.06%, or about 0.009% to about 0.06%, or about 0.009% to about 0.03% by total weight of the composition.

Compounds according to Formula I can also be obtained from natural sources. For example, a compound according to Formula I may be found in a botanical extract. Accordingly, the methods described herein may comprise applying to the sensitive skin a composition comprising a botanical extract.

In one or more embodiments, the botanical extract is an extract of a plant of the genus Acronychia. In further embodiments, the botanical extract is an extract of Acronychia acidula (also known as lemon aspen). In one or more embodiments, at least one compound of the above Formula I is present in the extract of Acronychia at a concentration equal to or greater than about 0.01 to about 30%, or about 0.1% to about 30%, or about 0.1 to about 20%, or about 1% to about 20%, or about 1% to about 10%, or about 1.5% to about 9%, or about 3% to about 9%, by weight of the Acronychia extract. In further embodiments, the extract of Acronychia acidula comprises 3-(4-farnesyloxyphenyl)-propionic acid in a concentration ranging from about 1% to about 10% by total weight of the extract.

Suitable extracts may be obtained using conventional methods including, but not limited to, direct extraction of material from the biomass by grinding, macerating, pressing, squeezing, mashing, centrifuging, and/or processes such as cold percolation, agitation/distillation, microwave assisted extraction, supercritical/subcritical CO₂ compressed gas extraction with or without polar modifiers, pressurized solvent extraction, accelerated solvent extraction, pressurized or normal hot water extraction, surfactant assisted pressurized hot water extraction, oil extraction, membrane extraction, Soxhlet extraction, the gold finger distillation/extraction and/or processes disclosed, for example, in U.S. Pat. Nos. 7,442,391, 7,473,435, and 7,537,791 to Integrated Botanical Technologies, LLC, incorporated herein by reference, and the like, or by other methods such as solvent extraction, and the like.

Any of a variety of solvents including polar solvents, non-polar solvents, or combinations of two or more thereof may be used in methods comprising solvent extraction. Suitable polar solvents include polar inorganic solvents such as water and the like, polar organic solvents such as alcohols and corresponding organic acids, for example C₁-C₈ alcohols including methanol, ethanol, propanol, butanol, and the like and organic acids, including acetic acid, formic acid, propanoic acid, and the like, polyols and glycols, including C₁-C₈ polyols/glycols and the like, and combinations of two or more thereof. Suitable non-polar solvents include non-polar organic solvents such as alkanes, including C₁-C₈ alkanes, cycloalkanes, including C₁-C₈ alkanes, alkyl ethers, including C₁-C₈ alkyl ethers, Petroleum ethers, ketones, including C₁-C₈ ketones, methylene chloride, ethyl acetate, xylene, toluene, chloroform, vegetable oil, mineral oil and the like. In another embodiment extraction may be obtained by non-polar solvents described above or supercritical fluid extraction with or without a polar modifier such as C₁-C₈ alcohols, water, C₁-C₈ polyols/glycols or C₁-C₈ organic acids.

In one or more embodiments, the extract comprises an extract of Acronychia acidula. In some embodiments, the extract of the invention comprises a combination of polar and non-polar extracts of Acronychia acidula fruit. In another embodiment, the extract of the invention comprises alcoholic or glycolic extracts of Acronychia acidula fruit.

In one or more embodiments, the extract is a polar extract. In further embodiments, the extract is a polar extract prepared using a polar solvent comprising water, C₁-C₈ alcohols, C₁-C₈ polyols, or C₁-C₈ glycols, or combinations of two or more thereof. In certain embodiments, the extract is extracted using one or more C₁-C₄ alcohols, C₁-C₄ polyols, and/or C₁-C₄ glycols. In one or more embodiments, the extract is prepared using a solvent comprising methanol, ethanol, or a combination thereof with or without presence of water. In further embodiments, the extract is a polar extract extracted from Acronychia acidula fruit using a combination of alcohol and water.

In one or more embodiments, the extract is a non-polar extract prepared using a non-polar solvent comprising one or more C₁-C₈ alkanes, C₁-C₈ cycloalkanes, C₁-C₈ alkyl ethers, C₁-C₈ alkyl esters and/or chloroform, more preferably one or more C₁-C₈ alkanes, C₁-C₈ alkyl esters and/or chloroform. In further embodiments, extract is a non-polar extract prepared using hexanes, ethyl acetate, chloroform, or mixtures of two or more thereof. In yet further embodiments, the extract is a non-polar extract prepared using ethyl acetate.

For example, an extract using the fruit of Acronychia acidula may be prepared by homogenizing the fruit in a blender for 30 seconds with denatured alcohol in equal part to fruit. The pulp can then mixed and stirred for another 24 hours at ambient temperature (22 to 26 degrees C.). Additional denatured alcohol may be added as needed to keep the pulp covered well in alcohol. The mixture then can then be gravity filtered, and the resulting filter cake washed with additional amounts of denatured alcohol. The total filtrate then may then be dried under reduced pressure to remove alcohol. The residue can then be freeze dried to obtain dry matter free of extraction solvent and water. The extraction may be repeated on the filter cake a few times with an extract yield of 5-7% regularly obtained from each extraction.

Another example of the preparation of Acronychia acidula fruit extract is as follows: 500 μm of freeze-dried fruits of Acronychia acidula may be sliced into approximately 5 mm cubes and soaked with 5 L of ethanol at a ratio of 1:10 (raw material to solvent) and stirred at room temperature for 12 hours. The suspension may then be filtered and resulting filtrate concentrated under low pressure to afford a concentrate. The concentrate can then further be dried by freeze-drying methods to obtain 325 μm of residual material called crude extract (65% yield). A sample of the crude extract, 200 μm, can then be taken up in 1 L ethanol and stirred at room temperature overnight. The mixture may then filtered and dried at reduced pressure and at low temperature to provide the extract.

The extract may be present in an amount of about 0.00001, 0.0001, 0.001, 0.01, 0.1, 0.2, 0.3, 0.4, 0.5, 0.75, 1, 1.5 or 2 to about 0.00005, 0.0005, 0.005, 0.05, 0.5, 1, 1.5, 2, 2.5, 3, 4 or 5 wt. % by total weight of the composition. In one or more embodiments, the extract is present in an amount of about 0.01 to about 5 wt. % by total weight of the composition. In further embodiments, the extract is present in an amount of about 0.01 to about 3 wt. % by total weight of the composition. In one or more embodiments, the extract is present in an amount of about 0.1 to about 3 wt. % by total weight of the composition. In further embodiments, the extract is present in an amount of about 0.2 to about 2.5 wt. % by total weight of the composition. In one or more embodiments, the extract is present in an amount of about 0.5 to about 2 wt. % by total weight of the composition. In further embodiments, the extract is present in an amount of about 0.5 to about 1.5 wt. % by total weight of the composition.

### Sensitive Skin

As used herein, the term “sensitive skin” refers to an individual whose skin is more prone to unpleasant or undesired sensations or reactions in response to stimuli that does not provoke such responses in most individuals. In one or more embodiments, the reactions of sensitive skin may be sensorial, such as burning, pain, pruritis, stinging, tingling, itching, tightness feeling and/or dry feeling. In some embodiments, the reactions of sensitive skin may be visual, such as erythema (redness), edema (swelling), peeling skin, and/or flaky patches. In one or more embodiments, the sensitive skin comprises skin of a subject who has a reaction condition or a history of a reaction condition selected from the group consisting of erythema (redness), edema (swelling), burning, pain, pruritis, stinging, tingling, itching, tightness feeling, dry feeling, scaling, tactile roughness, peeling skin, flaky patches and combinations thereof when exposed to a retinoid. In one or more embodiments, the sensitive skin comprises skin of a subject who has a condition or a history of a condition selected from the group consisting of atopic dermatitis, rosacea, seborrheic dermatitis, contact dermatitis, psoriasis, dry skin, flaky skin.

### Uses

The compositions/compounds described herein may be used in treating skin for treating acne, wrinkles, improving skin barrier function and/or lightening skin. In one or more embodiments, said treatment is for a subject who has a condition or a history of a condition selected from the group consisting of atopic dermatitis, rosacea, seborrheic dermatitis, psoriasis, dry skin, flaky skin.

As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

Compositions of the invention are suitable for improving the texture of skin or improving the firmness of skin, or any of the conditions/symptoms described below.

As used herein, “improving the texture of skin” means the smoothing of the surface of the skin to remove either bumps or crevasses on the skin surface.

As used herein, “improving the firmness of skin” means the enhancing of the firmness or elasticity of the skin, preventing the loss of firmness or elasticity of skin, or preventing or treating sagging, lax and loose skin.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “age spots” means a condition of the skin associated with discrete pigmentation, e.g., small areas of darker pigmentation that may develop on the face as well as the hands.

Signs of skin aging also include the presence of diminished skin thickness, and abnormal or diminished synthesis of collagen, glycosaminoglycans, proteoglycans, elastin, or glycoproteins including fibronectin. In one embodiment, the sign of aging is selected from the abnormal or diminished synthesis of collagen, glycosaminoglycans, proteoglycans, elastin, or glycoproteins including fibronectin. In another embodiment, the sign of skin aging is diminished synthesis of collagen or elastin.

Examples of skin aging that may be treated by topical use of the compositions of this invention include, but are not limited to, wrinkles on the skin. As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

As used herein, “topical use” and “topically applying” means directly laying on or spreading on the skin, hair, or nail, e.g., by use of the hands or an applicator such as a wipe.

The compositions are also suitable for treating or preventing acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that is not visually apparent on the surface of the skin with the naked eye (e.g., as a lesion).

The compositions of the invention are also suitable for treating or preventing rosacea. As used herein, “rosacea” means skin with persistent erythema with or without papules, pustules, or nodules.

The compositions of the invention are also suitable for reducing epidermal hyperkeratinization. Accordingly, the composition may be used for treatment or prevention of conditions characterized by hyperkeratinization, such as acne or warts.

The embodiments described herein may be combined in any suitable combination. For example, an exemplary embodiment pertains to a method of treating acne, wrinkles and/or lightening skin in a subject having a reaction condition or a history of a reaction condition selected from the group consisting of erythema, edema, burning, pain, pruritis, stinging, tingling, itching, tightness feeling, dry feeling, scaling, tactile roughness, peeling skin, flaky patches, and combinations thereof, the method comprising applying to the skin an extract of Acronychia acidula and/or 3-(4-farnesyloxyphenyl)-propionic acid. As will be discussed further below, the 3-(4-farnesyloxyphenyl)-propionic acid may be chemically synthesized, naturally occurring in a botanical extract, or chemically synthesized and added to a botanical extract. In one or more embodiments, the applying step comprises applying to the sensitive skin a composition comprising a botanical extract is an extract of a plant of the genus Acronychia. In further embodiments, the botanical extract is an extract of Acronychia acidula. In yet further embodiments, the extract is a polar extract. In one or more embodiments, the extract is present in an amount of about 0.1 to about 3 wt. % by total weight of the composition. In further embodiments, the extract is present in an amount of about 0.4 to about 1.5 wt. % by total weight of the composition.

The compositions described herein may be applied to any skin in need of treatment on the human body. For example, application may be made to any one or more of the skin of the face, neck, chest, back, arms, axilla, hands and/or legs. In certain preferred embodiments, the method comprises applying a compound of Formula I to skin of the face.

Any suitable method of applying the extract to the skin in need may be used in accordance with the present invention. For example, the extract may be applied directly from a package to the skin in need, by hand to the skin in need, or may be transferred from a substrate such as a wipe or mask, or a combination of two or more thereof. In other embodiments, the extract may be applied via a dropper, tube, roller, spray, patch or added to a bath or otherwise to water to be applied to the skin, and the like.

In one or more embodiments, the methods of the present invention further comprise the step of leaving the compound of Formula I in contact with the skin for period of time. For example, in certain preferred embodiments after application, the compound is left in contact with the skin for a period of about 15 minutes or greater. In certain more preferred embodiments, the extract is left in contact with the skin for about 20 minutes or greater, more preferably about 1 hour or greater.

In some embodiments, the method of the present invention comprises a regimen comprising applying the compound of Formula I to skin multiple times over a selected period of time. For example, in certain embodiments, the present invention provides a method of treating signs of aging comprising applying to skin in need of antiaging a composition comprising a compound of Formula I once or twice daily for at least 12 weeks, preferably at least 8 weeks and more preferably for at least 2 weeks.

### Compositions

Any suitable carrier may be used in the compositions of the present invention. Preferably, for a skin care composition, the carrier is a cosmetically-acceptable carrier. As will be recognized by those of skill in the art, cosmetically-acceptable carriers comprise carriers that are suitable for use in contact with the body, in particular the skin for antiaging applications, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 99.8% to about 98% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cP to about 200,000 cP. Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, makeup, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like.

The following are non-limitative examples of such carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentylene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99%.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid or dissolvable substrate (eg., a wipe, mask, pad, glove or strip).

The compositions of the present invention may further comprise any of a variety of additional cosmetically active agents, although they are preferably formulated to account for use on sensitive skin. Examples of suitable additional active agents include: additional skin lightening agents, darkening agents, anti-acne agents, shine control agents, antimicrobial agents such as anti-yeast agents, anti-fungal, and anti-bacterial agents, anti-inflammatory agents, anti-parasite agents, external analgesics, sunscreens, photo-protectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include hydroxy acids, benzoyl peroxide, D-panthenol, UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH₂, and other botanical extracts such as oat, aloe vera, Feverfew, Soy, Shiitake mushroom extracts, and derivatives and mixtures thereof.

In one embodiment, the compositions of the present invention are skin care compositions that comprise a compound of Formula I and at least one skin lightening active agent. Examples of suitable skin lightening active agents include, but are not limited to, tyrosinase inhibitors, melanin-inhibiting agents, melanosome transfer inhibiting agents including PAR-2 antagonists, exfoliants, sunscreens, retinoids, antioxidants, Tranexamic acid, skin bleaching agents, allantoin, opacifiers, talcs and silicas, zinc salts, and the like, and other agents as described in Solano et al. Pigment Cell Res. 2006, 19 (550-571). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavonoids, Licorice root extract, Mulberry root extract, Dioscorea coposita root extract, Saxifraga extract and the like, Ellagic acid, Salicylates and derivatives, Glucosamine and derivatives, Fullerene, Hinokitiol, Dioic acid, Acetyl glucosamine, Magnolignane, combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4alkylresorcinols such as 4-butyresorcinol (rucinol), 4-hexylresorcinol, phenylethyl resorcinol, 1 (2,4-dihydroxyphenyl)-3-(2,4-dimethoxy-3-methylphenyl)-Propane and the like and natural extracts enriched in resorcinols. Examples of salicylates include, but are not limited to, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol, retinaldehyde, retinoic acid, retinyl palmitate, isotretinoin, tazarotene, bexarotene and Adapalene. In certain preferred embodiments, the retinoid is retinol. Nevertheless, in one or more embodiments, the composition is essentially free of retinoids or retinoid precursors; and in further embodiments, is free of retinoids or retinoid precursors.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, and ascorbic acid and ascorbic acid derivatives (e.g., ascorbyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, feverfew, parthenolide-free feverfew, oat extracts, pomelo extract, wheat germ extract, Hesperidin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

A variety of other materials may also be present in the compositions of the present invention. These include, for example, chelating agents, humectants, opacifiers, conditioners, preservatives, fragrances and the like. The compositions may include surfactants, for example, those selected from the group consisting of anionic, non-ionics, amphoteric, cationic, or a combination of two or more thereof.

The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

In certain preferred embodiments, the present invention comprises applying a compound or composition of the invention via a substrate comprising such material. Any suitable substrate may be used in the present invention. Examples of suitable substrates and substrate materials are disclosed, for example, in U.S. Published Application Nos. 2005/022683 and 2009/0241242 which are incorporated herein by reference in their entirety. In certain preferred embodiments, the substrate is a wipe or a facial mask.

The composition and products containing such compositions of this invention may be prepared using methodology that is well known by an artisan of ordinary skill.

### Example 1: Composition Preparation

Two inventive compositions (Ex. 1-2) were prepared in the following manner using the amounts shown in Table 1 below. Ex. 1-2 contain 0.5 and 1 wt. % of lemon aspen extract, respectively. Purified water was added to a suitable sized beaker. Mixing was started using a propeller mixing blade and the water was heated to 75-80° C. Sepimax Zen™ was slowly added into the main batch and mixed until fully hydrated and free of visible particles, with the mixing speed increased as needed. The mixture was reheated to 75-80° C., then chlorphenesin and emulsifier were added and mixed until uniform and free of undissolved particles. Next, the solution was allowed to cool to 60-65° C., with the addition of glycerin, and mixed until uniform. Once the solution cooled to 55-60° C., Sepiplus™ 400 and Acronychia Acidula (lemon aspen) fruit extract (30% w/w in aqueous carrier solution) were added and mixed until uniform. Acronychia Acidula Fruit extract used in the examples typically contains between about 1% and 10% 3-(4-farnesyloxyphenyl)-propionic acid, with these samples containing 1.11% 3-(4-farnesyloxyphenyl)-propionic acid) by weight of the extract in the extract in carrier. Cooling continued to 40° C. while the solution was mixed at moderate speed. Then dimethicone, phenyoxyethanol, and ethylhexylglycerin were added and mixed until uniform. Water was added q.s. and the pH was adjusted to pH=5. The solution was mixed until uniform.

TABLE 1
Inventive Examples

| INCI | Ex. 1 | Ex. 2 |
|---|---|---|
| Acronychia Acidula Fruit Extract | 1.67 | 3.34 |
| (30% extract in carrier) | | |
| Emulsifier | 0.5 | 0.5 |
| Thickeners (Sepimax Zen ™ (INCI: Polyacrylate | 2.29 | 2.29 |
| Crosspolymer-6) and Sepiplus ™ 400 | | |
| (INCI: Polyacrylate-13 and Polyisobutene | | |
| and Polysorbate 20)) | | |
| Preservatives (phenoxyethanol, ethylhexyl | 1.00 | 1.00 |
| glycerin, chlorphenesin) | | |
| Skin Conditioning Agents (Glycerin, | 4.50 | 4.50 |
| Dimethicone) | | |
| Water | q.s to 100 | q.s to 100 |
| Sodium Hydroxide | q.s. to | q.s. to |
| | pH 5 | pH 5 |

### Example 2: Skin Irritation Model Clinical Study

Eight compositions were evaluated for the induction of clinical irritation using a Skin Cumulative Irritation patch test. Of the eight compositions, two were the inventive compositions Ex. 1 and 2 from above. Ex. 3-4 were two compositions which served as controls. Specifically, Ex. 3 was a positive control containing 0.5% sodium lauryl sulfate (SLS) in petrolatum. SLS serves as a positive control, as it is a known irritant (see e.g., Zhai, H. et al., A bioengineering study on the efficacy of a skin protectant lotion in preventing SLS-induced dermatitis, Skin Research and Technology 2000: 6: 77-80, see page 77). Ex. 4 was a negative control comprised of petrolatum. Petrolatum is known for use as a negative control, as evidenced in Uliasz, MD, A. and Lebwohl, MD M., Evaluation of sensory irritation caused by topical medications using a novel technique, International Journal of Dermatology 2010, 49, 269-27, see Abstract. Ex. 5-8 were commercially available compositions containing between 0 and 0.5% by weight of retinol.

60 subjects were recruited for this study, with 54 subjects completing the study. In the study, 0.2-0.3 grams of test composition was applied to a designated area on the back of each subject and fixed in place using a semi-occlusive patch (Professional Medical Products Patch) for 24 hours. Each composition was applied in this manner three times per week for a total of nine applications. Forty-eight to seventy-two hours after each application, a trained grader evaluated the test areas using the scale below in Table 2.

TABLE 2
Grading Scale

| Score | Description |
|---|---|
| 0 | No visible Reaction |
| 0.5 | Faint, minimal reaction |
| 1.0 | Erythema |
| 2.0 | Erythema, induration |
| 3.0 | Erythema, induration, vesicles |
| 4.0 | Severe reaction with erythema, induration, |
| | vesicles (may be weeping), postules |
| E | Indicates the presence of edema |

The cumulative irritation potential of a composition is the sum of nine application scores (21 days) for each test site for each subject. A grand total score for each composition was obtained by summing the 21 days totals for all subjects (Highest potential cumulative irritation score=1944). The cumulative irritation mean scores, standard deviation, minimum, median and maximum are shown in Table 3 below. The skin cumulative irritation mean scores were then compared by calculating levels, which were calculated from a mixed model with Tukey's HSD test for pairwise comparison. The testing hypothesis is that the mean change from baseline is equal between treatments. The resulting skin cumulative irritation scores and statistics are shown below in Table 4.

TABLE 3
Descriptive Statistics of the Skin Cumulative Irritation Score

| | | Overall | | Standard | | | |
|---|---|---|---|---|---|---|---|
| | Description | Sum | Mean | Deviation | Minimum | Median | Maximum |
| Ex. 1 | Composition | 0.00 | 0.00 | 0.00 | 0.00 | 0.00 | 0.00 |
| (Inv.) | Containing 0.5% | | | | | | |
| | Lemon Aspen | | | | | | |
| | Extract | | | | | | |
| Ex. 2 | Composition | 0.00 | 0.00 | 0.00 | 0.00 | 0.00 | 0.00 |
| (Inv.) | Containing 1% | | | | | | |
| | Lemon Aspen | | | | | | |
| | Extract | | | | | | |
| Ex. 3 | Positive Control | 73.50 | 1.36 | 1.88 | 0.00 | 0.00 | 7.00 |
| (Comp.) | (0.5% Sodium | | | | | | |
| | Lauryl Sulfate | | | | | | |
| | (SLS)) | | | | | | |
| Ex. 4 | Negative Control | 2.00 | 0.04 | 0.19 | 0.00 | 0.00 | 1.00 |
| (Comp.) | (Petrolatum) | | | | | | |
| Ex. 5 | Commercial Cream | 3.50 | 0.06 | 0.31 | 0.00 | 0.00 | 2.00 |
| (Comp.) | Containing 0.04% | | | | | | |
| | Retinol | | | | | | |
| Ex. 6 | Commercial Cream | 30.00 | 0.56 | 1.04 | 0.00 | 0.00 | 4.00 |
| (Comp.) | Containing 0.1% | | | | | | |
| | Retinol | | | | | | |
| Ex. 7 | Commercial Serum | 281.00 | 5.20 | 4.05 | 0.00 | 3.50 | 13.50 |
| (Comp.) | Containing 0.3% | | | | | | |
| | Retinol | | | | | | |
| Ex. 8 | Commercial Cream | 532.00 | 9.85 | 4.48 | 2.00 | 10.75 | 24.00 |
| (Comp.) | Containing 0.5% | | | | | | |
| | Retinol | | | | | | |

TABLE 4
Treatment Comparisons of the Skin Cumulative Irritation Score

| | | Est. | | | | |
|---|---|---|---|---|---|---|
| | Description | Mean | Levels | | | |
| Ex. 1 | Composition Containing | −0.00 | | | | D |
| (Inv.) | 0.5% Lemon Aspen Extract | | | | | |
| Ex. 2 | Composition Containing | −0.00 | | | | D |
| (Inv.) | 1% Lemon Aspen Extract | | | | | |
| Ex. 3 | Positive Control (0.5% | 1.36 | | | C | |
| (Comp.) | Sodium Lauryl Sulfate (SLS)) | | | | | |
| Ex. 4 | Negative Control | 0.04 | | | | D |
| (Comp.) | (Petrolatum) | | | | | |
| Ex. 5 | Commercial Cream | 0.06 | | | | D |
| (Comp.) | Containing 0.04% Retinol | | | | | |
| Ex. 6 | Commercial Cream | 0.56 | | | C | D |
| (Comp.) | Containing 0.1% Retinol | | | | | |
| Ex. 7 | Commercial Serum | 5.20 | | B | | |
| (Comp.) | Containing 0.3% Retinol | | | | | |
| Ex. 8 | Commercial Cream | 9.85 | A | | | |
| (Comp.) | Containing 0.5% Retinol | | | | | |

The results of inventive Ex. 1-2 are surprising given that the overall sum was equivalent to the negative control petrolatum, which is well-known as a very gentle skin protectant. Even the sample with the lowest concentration of retinol (0.04% in Ex. 5) was higher than the negative control. Also, lemon aspen extract (comprising the compound 3-(4-farnesyloxyphenyl)-propionic acid) has retinol-like activity, as evidenced by its mechanism of action through the retinol agonist receptor (RAR) pathway (see e.g., U.S. Patent Publication No. 20200030226A1). Retinol and retinoids in general, are typically not considered to gentle to the skin, and in fact are known to cause local irritation and erythema of the skin (Kim, B. et al., The mechanism of retinol-induced irritation and its application to anti-irritant development, Toxicology Letters 146 (2003), 65-73, see abstract). It is therefore surprising that 0.5% and 1% w/w lemon aspen extract compositions were so gentle to the skin, even though the lemon aspen extract functions using the same mechanism of action.

### Example 3: Tolerability Clinical Study in Subjects with Sensitive Skin

Three compositions were evaluated for clinical tolerability in a single-center, 12-week, 3-cell, evaluator-blinded, full-face, randomized study. Of the three compositions, two were the inventive compositions Ex. 1 and 2 from above. The third composition was a Commercial Cream Containing 0.1% Retinol.

Subjects were screened and enrolled to ensure completion of at least 30 subjects in each of the three (3) cells. The target population was 35- to 60-year-old female subjects of Fitzpatrick Skin Types I-IV who had moderate mottled hyperpigmentation, moderate fine lines/wrinkles, and moderate overall photodamage of their facial skin. At least 50 percent of the subjects in treatments groups Ex 1 and 2 had self-perceived mildly to moderately sensitive facial skin using the scale in Table 5. For the Commercial Cream Containing 0.1% Retinol treatment, all subjects had self-perceived normal skin (i.e., not sensitive at all). Retinol was not tested on subjects having sensitive skin because of the known irritation and erythema that it causes.

TABLE 5
Self-perceived sensitivity grading scale

| Scale | Grade |
|---|---|
| 5 | Extremely sensitive |
| 4 | Very sensitive |
| 3 | Moderately sensitive |
| 2 | Mildly sensitive |
| 1 | Not at all sensitive |

Starting at Visit 1, the subjects washed their face once in the morning and once at night using a particular commercially available cleanser. Starting at Visit 2, subjects used the assigned treatment once in the morning and once at night after using the provided cleanser. Additionally, the subjects applied a particular commercially available sunscreen, 5-10 minutes after applying the facial moisturizer. Subjects were only permitted to use their normal cosmetic makeup products which were approved in Visit 1. On study visit days, subjects removed all leave on products with the provided cleanser at least 30 mins prior to the visit and at most 2 hours prior to the visit. The study consisted of 6 visits over 12 weeks with visits scheduled at Screening (Visit 1), Baseline (Week 0; Visit 2), Week 1 (Visit 3), Week 2 (Visit 4), Week 4 (Visit 5), and Week 12 (Visit 6). The Commercial Cream Containing 0.1% Retinol treatment group was not assessed at Week 1.

The principal investigator (PI), an expert in clinical grading of skin having 15 years of experience in this field, assessed each subject's facial skin for the tolerance parameters of erythema, edema, and dryness/scaling using a 0-3 grading scale with half points allowed, pursuant to the grading scale in Table 6.

TABLE 6
Grading scales for Tolerance parameters (clinical)
Burning/Stinging

| | |
|---|---|
| 0 = None | No burning/stinging of the treatment area |
| 1 = Mild | Slight burning/stinging sensation of the treatment |
| | area; not really bothersome |
| 2 = Moderate | Definite warm burning/stinging of the treatment area |
| | that is somewhat bothersome |
| 3 = Severe | Hot burning/marked stinging sensation of the treatment |
| | area that causes definite discomfort and may interrupt |
| | daily activities and/or sleep |

Itching

| | |
|---|---|
| 0 = None | No itching of the treatment area |
| 1 = Mild | Slight itching sensation of the treatment area; not really |
| | bothersome |
| 2 = Moderate | Definite itching of the treatment area that is somewhat |
| | bothersome |
| 3 = Severe | Marked itching sensation of the treatment area that |
| | causes definite discomfort and may interrupt daily |
| | activities and/or sleep |

Tightness/Dry Feeling

| | |
|---|---|
| 0 = None | No skin tightness of the treatment area |
| 1 = Mild | Slight, but definite tightness or dry feeling of the |
| | treatment area |
| 2 = Moderate | Definite tightness or dry feeling of the treatment area |
| | that is somewhat bothersome |
| 3 = Severe | Marked tightness or dry feeling of the treatment area |
| | that causes definite discomfort |

The PI also questioned the subjects for the tolerance parameters of Burning, Stinging, Itching, and Tightness/dry feeling using a 0-3 grading scale using whole points only pursuant to the grading scale in Table 7.

TABLE 7
Grading scales for Tolerance Parameters (subjective).
Burning/Stinging

| | |
|---|---|
| 0 = None | No burning/stinging of the treatment area |
| 1 = Mild | Slight burning/stinging sensation of the treatment |
| | area; not really bothersome |
| 2 = Moderate | Definite warm burning/stinging of the treatment area |
| | that is somewhat bothersome |
| 3 = Severe | Hot burning/marked stinging sensation of the treatment |
| | area that causes definite discomfort and may interrupt |
| | daily activities and/or sleep |

Itching

| | |
|---|---|
| 0 = None | No itching of the treatment area |
| 1 = Mild | Slight itching sensation of the treatment area; not really |
| | bothersome |
| 2 = Moderate | Definite itching of the treatment area that is somewhat |
| | bothersome |
| 3 = Severe | Marked itching sensation of the treatment area that |
| | causes definite discomfort and may interrupt daily |
| | activities and/or sleep |

Tightness/Dry Feeling

| | |
|---|---|
| 0 = None | No skin tightness of the treatment area |
| 1 = Mild | Slight, but definite tightness or dry feeling of the |
| | treatment area |
| 2 = Moderate | Definite tightness or dry feeling of the treatment area that |
| | is somewhat bothersome |
| 3 = Severe | Marked tightness or dry feeling of the treatment area that |
| | causes definite discomfort |

The tolerance scores above were evaluated using a Freidman's Test, with a nonparametric Repeated Measures ANOVA with Dunn's as a post hoc test to determine the level of significance from baseline at each timepoint. The mean change from baseline for each tolerance parameter are shown in Tables 8 and 9 below.

TABLE 8
Mean change from baseline by Tolerance
Parameter over 12 weeks.

Mean change from baseline

| | | | |
|---|---|---|---|
| Tolerance | | | Commercial Cream |
| parameter/ | | | Containing |
| Week | Ex. 1 | Ex. 2 | 0.1% Retinol |

Erythema

| | | | |
|---|---|---|---|
| Week 1 | −0.018 | 0.141 | NA |
| Week 2 | 0.344 | 0.40 | 0.323 |
| Week 4 | 0.203 | 0.355 | 0.155 |
| Week 12 | 0.205 | 0.355 | 0.387 |

Edema

| | | | |
|---|---|---|---|
| Week 1 | 0.000 | 0.000 | NA |
| Week 2 | 0.016 | 0.017 | 0.016 |
| Week 4 | 0.016 | 0.016 | 0.017 |
| Week 12 | 0.000 | 0.000 | 0.000 |

Dryness/Scaling

| | | | |
|---|---|---|---|
| Week 1 | −0.214 | −0.219 | NA |
| Week 2 | −0.234 | −0.033 | −0.048 |
| Week 4 | −0.250 | −0.145 | 0.034 |
| Week 12 | −0.172 | −0.048 | 0.000 |

TABLE 9
Mean change from baseline by Subjective
Tolerance Parameter over 12 weeks.

Mean change from baseline

| | | | |
|---|---|---|---|
| Tolerance | | | Commercial Cream |
| parameter/ | | | Containing |
| Week | Ex. 1 | Ex. 2 | 0.1% Retinol |

Burning

| | | | |
|---|---|---|---|
| Week 1 | 0.000 | 0.219 | NA |
| Week 2 | 0.063 | 0.067 | 0.129 |
| Week 4 | 0.031 | 0.065 | 0.069 |
| Week 12 | 0.031 | 0.065 | 0.032 |

Stinging

| | | | |
|---|---|---|---|
| Week 1 | 0.071 | 0.125 | NA |
| Week 2 | 0.250 | 0.200 | 0.258 |
| Week 4 | 0.125 | 0.129 | 0.103 |
| Week 12 | 0.000 | 0.097 | 0.000 |

Itching

| | | | |
|---|---|---|---|
| Week 1 | 0.000 | 0.000 | NA |
| Week 2 | 0.000 | 0.000 | 0.032 |
| Week 4 | 0.063 | −0.032 | 0.000 |
| Week 12 | 0.031 | 0.032 | 0.065 |

Tightness/Dry Feeling

| | | | |
|---|---|---|---|
| Week 1 | −0.393 | −0.531 | NA |
| Week 2 | −0.031 | −0.433 | 0.065 |
| Week 4 | −0.250 | −0.581 | −0.069 |
| Week 12 | −0.313 | −0.548 | −0.065 |

These data show no statistically significant differences in net change from baseline between cells at any timepoint for all endpoints, indicating that the treatments were non-irritating. As discussed above, lemon aspen extract (comprising the compound 3-(4-farnesyloxyphenyl)-propionic acid) has retinol-like activity, as evidenced by its mechanism of action through the retinol agonist receptor (RAR) pathway, and retinoids are generally known to not be well tolerated. Therefore it is surprising that lemon aspen extract was very well tolerated in the self-perceived sensitive skin population. As can also be seen from the results, the samples containing lemon aspen extract were as non-irritating or less irritating in individuals having sensitive skin as compared to the retinol-containing composition in individuals having normal skin.

### Example 4: Instrument Testing—Cutometer

Three (3) elasticity measurements were performed using an elasticity characterization instrument sold under the tradename CUTOMETER MPA 580 (Courage+Khazaka electronic, Germany) on the left cheek area of the subjects from EXAMPLE 3, at each timepoint. The Commercial Cream Containing 0.1% Retinol treatment group was not assessed at Week 1. The probe was repositioned for each measurement using 450 mbar of pressure, a 2-mm diameter probe aperture, 5 seconds of suction, and 5 seconds relaxation time. Each check-area measurement was approximately 2 cm below the cheekbone and avoided any blemishes or hyperpigmented spots.

The probe was placed perpendicularly on the skin in full contact with the skin while not exerting too much force that would deform the skin. The viscoelastic behavior of the skin was described through a deformation curve, with skin deformation (amplitude in mm) measured as a function of time. The following absolute parameters (measured in mm) were calculated from a skin deformation curve:
Ua/Uf (R2)—the ratio between final retraction and maximal deformation (i.e., gross elasticity of the skin, including viscous deformation)
Ur/Uf (R7)—the ratio of immediate retraction to total deformation (i.e., biological elasticity)
Ur/Ue (R5)—the ratio of immediate retraction to immediate deformation (i.e., net-elasticity of the skin without viscous deformation)

The results were evaluated using a Repeated Measures ANOVA with Dunn's as a post hoc test to determine the level of significant changes from baseline between the treatments at each timepoint. The mean change from baseline for each skin elasticity parameter are shown in Tables 10 below.

TABLE 10
Mean change from baseline for Cutometer
(R7, R5, and R2) over 12 weeks

| | Mean change from baseline | | |
|---|---|---|---|
| Cutometer | | | Commercial Cream |
| parameter/ | | | Containing |
| Week | Ex. 1 | Ex. 2 | 0.1% Retinol |

R7

| | | | |
|---|---|---|---|
| Week 1 | 0.007 | 0.035 | NA |
| Week 2 | 0.019 | 0.041 | 0.026 |
| Week 4 | 0.021 | 0.042 | 0.019 |
| Week 12 | 0.029 | 0.044 | 0.031 |

R5

| | | | |
|---|---|---|---|
| Week 1 | 0.000 | 0.050 | NA |
| Week 2 | 0.022 | 0.091 | 0.040 |
| Week 4 | 0.029 | 0.060 | 0.033 |
| Week 12 | 0.038 | 0.075 | 0.053 |

R2

| | | | |
|---|---|---|---|
| Week 1 | 0.005 | 0.039 | NA |
| Week 2 | 0.020 | 0.053 | 0.037 |
| Week 4 | 0.034 | 0.061 | 0.036 |
| Week 12 | 0.052 | 0.074 | 0.059 |

Cutometer R2 values show an improvement in gross elasticity (the total recovery over the total deformation) compared to Baseline for all Cells across all timepoints, with continued improvement through Week 12 for all Cells.

Cutometer R5 values show an improvement in net elasticity (the immediate recovery over the immediate deformation) compared to Baseline for all Cells, with little change in Cells B and C from Week 2 through Week 12 and a small decrease in this improvement for Cell A from Week 2 through Week 12.

Cutometer R7 values show an improvement in elasticity (the immediate recovery over the total deformation) compared to Baseline for all Cells. The only statistically significant differences between Cells were between Cell A and Cell B at Week 1 (p<0.05).

Cutometer values for R2, R5, and R7 show improvements in gross elasticity (the total recovery over the total deformation), net elasticity (the immediate recovery over the immediate deformation) and elasticity (the immediate recovery over the total deformation), respectively when compared to baseline for all treatments across all timepoints. Therefore, the 0.5 and 0.1% Lemon Aspen compositions (Ex. 1-2) show efficacy in improving skin elasticity, which is indicated for treating signs of aging.

Additionally, the results of three parameters of lemon aspen, R2, R5, and R7, over 12 weeks showed a similar level of improvement in sensitive skin as 0.1% retinol did in normal skin. This demonstrates the efficacy of lemon aspen as an anti-aging treatment which is gentle enough for sensitive skin, and that the efficacy is comparable to retinol in normal skin.

### Example 5: Instrument Testing—Erythema Index

Erythema Index measurements were obtained using a DSM II Colorimeter (Cortex Technologies, Hadsund, Denmark). Measurements were performed on the left cheek area and center of the forehead of the subjects in EXAMPLES 3-4.

The results were evaluated using a Repeated Measures ANOVA with Dunn's as a post hoc test to determine the level of significant changes from baseline between treatments at each timepoint. The mean change from baseline for erythema index from Cheek measurements are shown in Table 11 below.

TABLE 11
Mean change from baseline for erythema
index measurements over 12 weeks

| | |
|---|---|
| Erythema Index | Mean change from baseline |

| measurement/Week | Ex. 1 | Ex. 2 |
|---|---|---|
| Week 1 | 0.029 | 0.481 |
| Week 2 | −0.479 | −0.708 |
| Week 4 | −1.558 | −1.508 |
| Week 12 | −1.811 | −1.471 |

Erythema index data show small, incremental decreases in Erythema from Week 2 through Week 12 for both lemon aspen compositions. There were no statistically significant differences between treatments.

DSM II Erythema Index values from the Cheek show small, incremental decreases in Erythema from Week 2 through Week 12 for both Cells A and B. There were no statistically significant differences between Cells.

DSM II Erythema Index values from the Forehead show small, incremental decreases in Erythema from Week 2 through Week 12 for both Cells A and B. There were no statistically significant differences between Cells.

There was no skin irritation manifested by skin redness measured by erythema index. These results confirm the observations in the above patch test study, that the compositions are non-irritating on sensitive skin.

### Example 5: Instrument Testing—Collagen Crosslinks

Three (3) fluorescence measurements (i.e., three (3) sets of scans) were performed using a SkinSkan spectrofluorometer (Horiba Jovin Yvon, Edison, NJ) on the left cheek area and the center of the forehead of the subjects from EXAMPLES 3-5. One SkinSkan measurement was a set of three scans-two excitation scans (1:270 nm-320 nm, emission at 345 nm, 2:290 nm-360 nm, emission at 390 nm) followed by a sync scan (315 nm-401 nm). Analysis of the fluorescence spectrum provides information on skin fluorophores such as tryptophan, collagen crosslinks. The Commercial Cream Containing 0.1% Retinol treatment group was not assessed at Week 1.

The results were evaluated using a Repeated Measures ANOVA with Dunn's as a post hoc test to determine the level of significant changes from baseline between treatments at each timepoint. The mean change from baseline for fluorescence intensity from collagen crosslinks are shown in Table 12 below.

TABLE 12
Mean change from baseline for collagen
crosslinks fluorescence over 12 weeks

Mean change from baseline

| | | | Commercial Cream |
|---|---|---|---|
| | | | Containing |
| Fluorescence | Ex. 1 | Ex. 2 | 0.1% Retinol |
| Crosslinks Cheek | 0.92 | 0.67 | 0.74 |
| Week 12 | | | |
| Crosslinks Forehead | 0.14 | 0.07 | 0.13 |
| Week 12 | | | |

Ex. 1 and 2 each show a statistically significant improvement in collagen crosslinks at Week 12 compared to baseline. Such improvement in collagen crosslinks is indicated for treating signs of aging. Therefore, the 0.5 and 0.1% Lemon Aspen compositions (Ex. 1-2) show efficacy in treatment for signs of skin aging.

Additionally, the results for Ex. 1-2 showed a similar level of improvement in sensitive skin as 0.1% retinol did in normal skin. This demonstrates the efficacy of lemon aspen as an anti-aging treatment which is gentle enough for sensitive skin, and that the efficacy is comparable to retinol in normal skin.

1. A method of treating sensitive skin, the method comprising applying to the sensitive skin an a compound of Formula I: 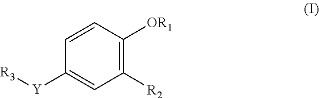
wherein:
R₁ is selected from the group consisting of C₁-C₂₀ alkyl, C₂-C₂₀ alkenyl, C₂-C₂₀ alkynyl, and C₃-C₈ cycloalkyl or aryl;
R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
R₃ is selected from —CO₂H, —CO₂R₄ or an isosteric equivalent of a carboxy group, wherein R₄ is C₁-C₆ alkyl, C₂-C₆ alkenyl, C₃-C₈ cycloalkyl or aryl; and
Y is —(CH₂—CH₂)—, —(CH═CH)—, or —(C═C)—;
or a cosmetically acceptable salt thereof.

2. The method of claim 1 wherein R₁ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R₄ is C₁-C₆ alkyl, or an isosteric equivalent of a carboxy group; and Y is —(CH₂—CH₂)— or —(CH═CH)—.

3. The method of claim 1, wherein R₁ is selected from the group consisting of C₅-C₁₆ alkenyl; and R₂ is selected from the group consisting of hydrogen or —OC₁-C₃ alkyl.

4. The method of claim 1, wherein the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof.

5. The method of claim 4, wherein the compound of Formula I is 3-(4-farnesyloxyphenyl)-propionic acid.

6. The method of claim 1, wherein the applying step comprises applying to the sensitive skin the compound of Formula I, and the compound of Formula I is contained in a botanical extract.

7. The method of claim 6, wherein said botanical extract is an extract of a plant of the genus Acronychia.

8. The method of claim 7, wherein said botanical extract is an extract of Acronychia acidula.

9. The method of claim 6, wherein said extract is a polar extract.

10. The method of claim 6, wherein the extract is present in an amount of about 0.1 to about 3 wt. % by total weight of the composition.

11. The method of claim 1, wherein the concentration of the compound of Formula I is present in an amount ranging from about 0.0001% to about 1% by total weight of the composition.

12. The method of claim 1, wherein the sensitive skin comprises skin of a subject who has a reaction condition or a history of a reaction condition selected from the group consisting of erythema, edema, burning, pain, pruritis, stinging, tingling, itching, tightness feeling, dry feeling, scaling, tactile roughness, peeling skin, flaky patches and combinations thereof when exposed to a retinoid.

13. The method of claim 1, wherein the sensitive skin comprises skin of a subject who has a condition or a history of a condition selected from the group consisting of atopic dermatitis, rosacea, seborrheic dermatitis, contact dermatitis, psoriasis, dry skin, flaky skin.

14. The method of claim 1, wherein the method of treating skin is a method of treating acne, wrinkles, and/or lightening skin in a subject who has a condition or a history of a condition selected from the group consisting of atopic dermatitis, rosacea, seborrheic dermatitis, psoriasis, dry skin, flaky skin.

15. A method of treating acne, wrinkles and/or lightening skin in a subject having a reaction condition or a history of a reaction condition selected from the group consisting of erythema, edema, burning, pain, pruritis, stinging, tingling, itching, tightness feeling, dry feeling, scaling, tactile roughness, peeling skin, flaky patches, and combinations thereof, the method comprising applying to the skin 3-(4-farnesyloxyphenyl)-propionic acid.

16. The method of claim 15, wherein the applying step comprises applying to the sensitive skin a composition comprising a botanical extract is an extract of a plant of the genus Acronychia.

17. The method of claim 16, wherein said botanical extract is an extract of Acronychia acidula.

18. The method of claim 17, wherein said extract is a polar extract.

19. The method of claim 16, wherein the extract is present in an amount of about 0.1 to about 3 wt. % by total weight of the composition.

20. The method of claim 15, wherein the concentration of the 3-(4-farnesyloxyphenyl)-propionic acid is in an amount ranging from about 0.0001% to about 1% by total weight of the composition.
